(19) United States
(12) Patent Application Publication
(10) Pub. No.: US 2017/0020444 A1
(43) Pub. Date: Jan. 26, 2017

(54) SYSTEMS AND METHODS FOR MODIFYING PHYSIOLOGICAL PARAMETERS AND DISEASE

(71) Applicant: Yair Lurie, Santa Barbara, CA (US)

(72) Inventor: Yair Lurie, Santa Barbara, CA (US)

(21) Appl. No.: 15/218,999

(22) Filed: Jul. 25, 2016

Related U.S. Application Data
(60) Provisional application No. 62/197,069, filed on Jul. 26, 2015.

(51) Int. Cl. A61B 5/00; A61B 5/01; A61B 5/0402; A61B 5/16; A61B 5/0488; A61B 5/08; A61B 5/145; A61B 5/021; A61B 5/0476

(52) U.S. Cl. CPC A61B 5/486; A61B 5/0024; A61B 5/7475; A61B 5/7405; A61B 5/742; A61B 5/7455; A61B 5/021; A61B 5/01; A61B 5/0402; A61B 5/0476; A61B 5/0488; A61B 5/08; A61B 5/14542; A61B 5/681; A61B 5/6898; A61B 5/165; A61B 5/4818; A61B 5/168; A61B 5/002; A61B 5/0022

(57) ABSTRACT

Provided herein are systems and methods for setting a physiological threshold value in a user interface for a user behavior-modification system; monitoring a physiological parameter corresponding to the physiological threshold value; and notifying the user with a notification when the user exceeds the physiological threshold value. A portion of the user behavior-modification system can be resident in a memory of a first network-connected electronic device configured with a physiological sensor. The physiological sensor can be configured to physiologically couple to the user for monitoring a physiological parameter thereof. Monitoring the physiological parameter can include alerting the user when the user exceeds the physiological threshold value. Notifying the user enables the user to modify behavior to bring the physiological parameter below the physiological threshold value. The notification can be provided through a vibrator, a speaker, or a display screen of the first electronic device or a second network-connected electronic device.

### CROSS-REFERENCE

This application claims the benefit of U.S. Provisional Patent Application No. 62/197,069, titled “Mobile Blood Pressure Control System,” filed Jul. 26, 2015, which application is incorporated herein by reference in its entirety.

### FIELD

This application includes systems and methods for psychophysiology, biofeedback, and auto-regulation that provide feedback to a user thereof in order to gain control of voluntary and involuntary physiological activity.

### BACKGROUND

In view of increasing rates of comorbid disease populations, rising healthcare costs, and shortages in healthcare providers, patients would benefit from a disease management solution that allows them to take more control over their own health while receiving remote support from caregivers. A solution that allows patients to self-care while also allowing healthcare providers to remotely monitor and care for patients with chronic disease(s) would reduce the cost of healthcare and cope with the shortage of medical specialists. Provided herein are systems and methods for modifying physiological parameters and disease that address the foregoing needs.

### SUMMARY

Provided herein in some embodiments are systems and methods for setting a physiological threshold value in a user interface for a user behavior-modification system; monitoring a physiological parameter corresponding to the physiological threshold value; and notifying the user with a notification when the user exceeds the physiological threshold value in an event. At least a portion of the user behavior-modification system can be resident in a memory of a first network-connected electronic device configured with a physiological sensor. The physiological sensor can be configured to physiologically couple to the user for monitoring a physiological parameter of the user. Monitoring the physiological parameter corresponding to the physiological threshold value can include alerting the user when the user exceeds the physiological threshold value. Notifying the user with a notification when the user exceeds the physiological threshold value in an event enables the user to modify behavior to bring the physiological parameter below the physiological threshold value. The notification can be provided through a vibrator, a speaker, or a display screen of the first electronic device or a second network-connected electronic device.

These and other features of the concepts provided herein may be better understood with reference to the following drawings, description, and appended claims.

### DRAWINGS

FIG. 1A provides a user with a network-connected electronic device including a behavior-modification system in accordance with some embodiments while allowing a remote care team access to the user's data.

FIG. 1B provides a user with a network-connected electronic device including a behavior-modification system in accordance with some embodiments while allowing a remote care team access to the user's data.

FIG. 2 provides a user interface on an electronic device including a behavior-modification system in accordance with some embodiments.

FIG. 3 provides electronic devices including notifications provided by a behavior-modification system in accordance with some embodiments.

FIG. 4 provides a training module of a behavior-modification system in accordance with some embodiments.

FIG. 5 provides a self-assessment questionnaire of a behavior-modification system in accordance with some embodiments.

FIG. 6A provides a schematic illustrating a method of a behavior-modification system for providing notifications such as biofeedback notifications in accordance with some embodiments.

FIG. 6B provides a schematic illustrating a method of a behavior-modification system for providing notifications such as biofeedback notifications in accordance with some embodiments.

FIG. 6C provides a schematic illustrating a method of a behavior-modification system for providing notifications such as biofeedback notifications in accordance with some embodiments.

FIG. 7 provides one or more computing systems in accordance with some embodiments.

### DESCRIPTION

Before some particular embodiments are provided in greater detail, it should be understood that the particular embodiments provided herein do not limit the scope of the concepts provided herein. It should also be understood that a particular embodiment provided herein can have features that can be readily separated from the particular embodiment and optionally combined with or substituted for features of any of a number of other embodiments provided herein.

Regarding terminology used herein, it should also be understood the terminology is for the purpose of describing some particular embodiments, and the terminology does not limit the scope of the concepts provided herein. Unless indicated otherwise, ordinal numbers (e.g., first, second, third, etc.) are used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. It should also be understood that, unless indicated otherwise, any labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or directions. It should also be understood that the singular forms of “a,” “an,” and “the” include plural references unless the context clearly dictates otherwise.

Unless defined otherwise, all technical and scientific terms used herein have the same meaning as commonly understood those of ordinary skill in the art.

In view of increasing rates of comorbid disease populations, rising healthcare costs, and shortages in healthcare providers, patients would benefit from a disease management solution that allows them to take more control over their own health while receiving remote support from caregivers. A solution that allows patients to self-care while also allowing healthcare providers to remotely monitor and care for patients with chronic disease(s) would reduce the cost of healthcare and cope with the shortage of medical specialists. Provided herein are systems and methods for modifying physiological parameters and disease that address the foregoing needs.

Provided herein in some embodiments are systems and methods for setting a physiological threshold value in a user interface for a user behavior-modification system; monitoring a physiological parameter corresponding to the physiological threshold value; and notifying the user with a notification when the user exceeds the physiological threshold value in an event. At least a portion of the user behavior-modification system can be resident in a memory of a first network-connected electronic device configured with a physiological sensor. The physiological sensor can be configured to physiologically couple to the user for monitoring a physiological parameter of the user. Monitoring the physiological parameter corresponding to the physiological threshold value can include alerting the user when the user exceeds the physiological threshold value. Notifying the user with a notification when the user exceeds the physiological threshold value in an event enables the user to modify behavior to bring the physiological parameter below the physiological threshold value. The notification can be provided through a vibrator, a speaker, or a display screen of the first electronic device or a second network-connected electronic device.

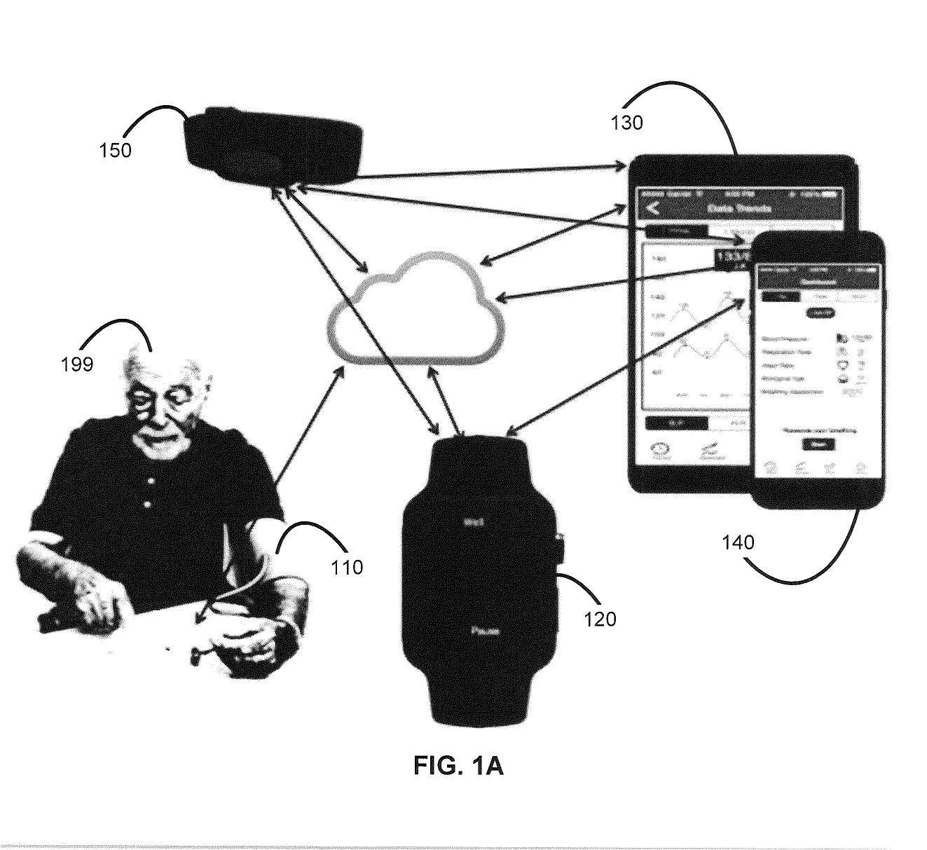

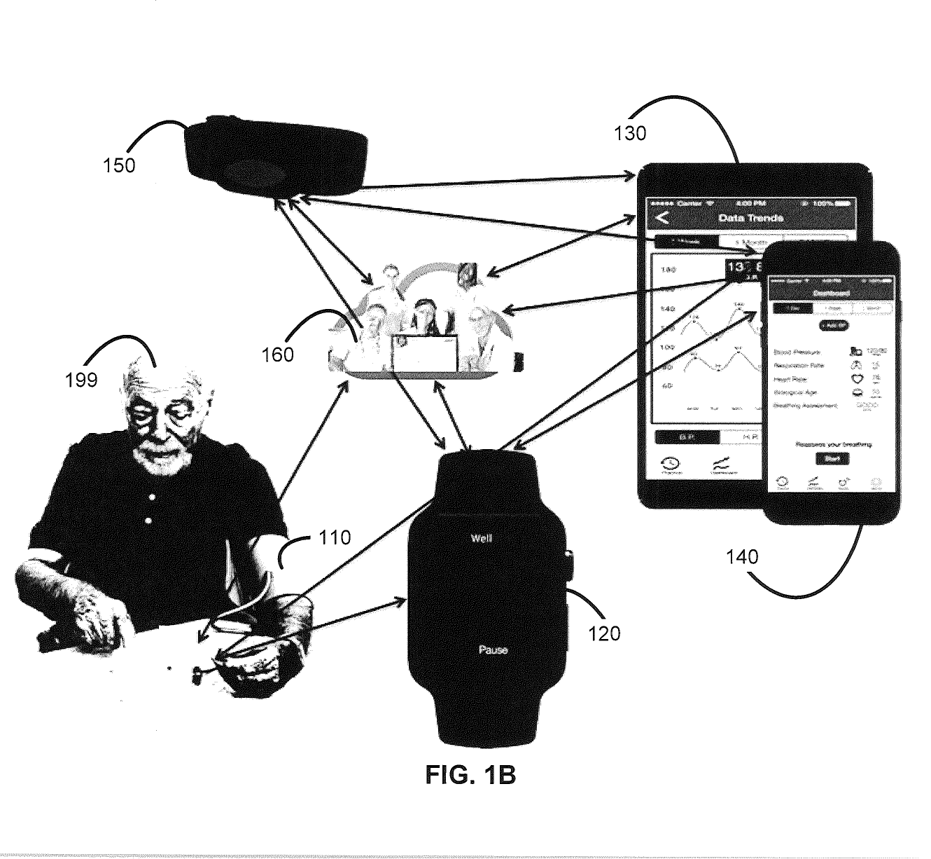

FIG. 1A provides a user with a network-connected electronic device including a behavior-modification system in accordance with some embodiments while allowing a remote care team access to the user's data.

As shown, the user 199 can have hypertension or some other chronic disease that requires a first physiological sensor device 110 such as a blood pressure device to take blood pressure measurements. The blood pressure device can be a smart blood pressure device configured to communicate using Wi-Fi, Bluetooth®, and/or a cellular network protocol to transmit blood pressure results to a secure database or directly to another electronic device selected from a smart watch 120, a smart tablet 130, a phone 140, and a second physiological sensor device 150. The second physiological sensor device can be a smart heart rate sensor, a smart respiration sensor, a smart weight scale, or the like that can also be configured to communicate physiological measurements via Wi-Fi, Bluetooth®, or a cellular network protocol to a secure database or yet another electronic device such as the foregoing electronic devices.

A smart watch displaying real-time heart rate variability data can be worn by the user in order to get feedback on heart rate variability levels such as low heart rate variability resulting from stress, as well as high heart rate variability indicating balanced heart rate functioning. The smart watch has an option to include built-in physiological sensors such as a heart rate sensor that calculates heart rate variability and that also monitors other physiological readings such as temperature, pulse oximetry, and blood pressure. The smart watch can be configured to transmit physiological measurements and other data to a cloud-based database. The smart watch can also be configure to communicate wirelessly to the smart tablet, the smart phone, or similar devices that can display readings from the first physiological device, the second physiological device, and the smart watch, which enables users to keep track of blood pressure readings and other vital sign data allowing for self-monitoring. Real-time heart rate variability monitoring and breathing exercise capabilities are also provided as training modules to teach the user diaphragmatic breathing.

FIG. 1B provides a user with a network-connected electronic device including a behavior-modification system in accordance with some embodiments while allowing a remote care team access to the user's data.

As shown, FIG. 1B provides all the components of FIG. 1A but further includes one or more health care professionals of a remote care team 160, including one or more nurses, doctors, and coaches with the ability to remotely monitor any one or more of the users' blood pressure, breathing rate, heart rate variability, weight, and other physiological measurements in order to monitor and manage the users' blood pressure and overall health.

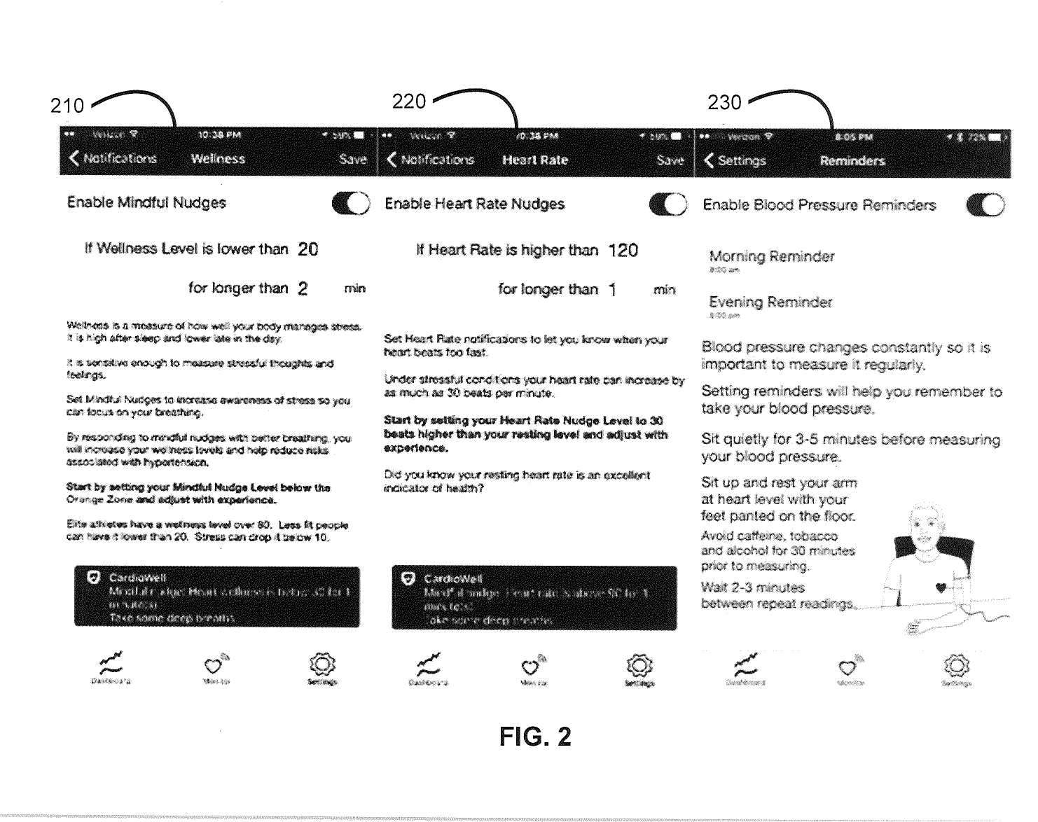

FIG. 2 provides a user interface on an electronic device including a behavior-modification system in accordance with some embodiments.

As shown in FIG. 2, the user interface can be part of an application that runs on a smart device that, in turn, enables the user to configure a variety of parameters to control the transmission of notifications, “nudges,” and alerts to the user through a smart watch, smart tablet, or smart phone. As shown in a wellness window 210 of the user interface, the user has an option to configure a notification when heart rate variability reaches a certain high or low threshold value. In this example it displays the ability for the user to configure a mindful nudge/notification when their rMSSD (wellness) falls below a value of 20 for longer than 2 minutes. This will result in a notification occurring if the user's HRV rMSSD value falls below 20 for 2 minutes. The user has the ability to configure the notification to any value and for any length of time. This provides a method to enable heart rate variability biofeedback. As shown in a heart rate window 220 of the user interface, the user has an ability to configure notifications based on heart rate. This allows the user to program a notification whenever the heart rate increases over a certain pulse rate for a certain period of time. As shown in a reminder window 230 of the user interface, the user also has the ability to set blood pressure reminders. Other screens or windows provide the user the ability to set medication reminders.

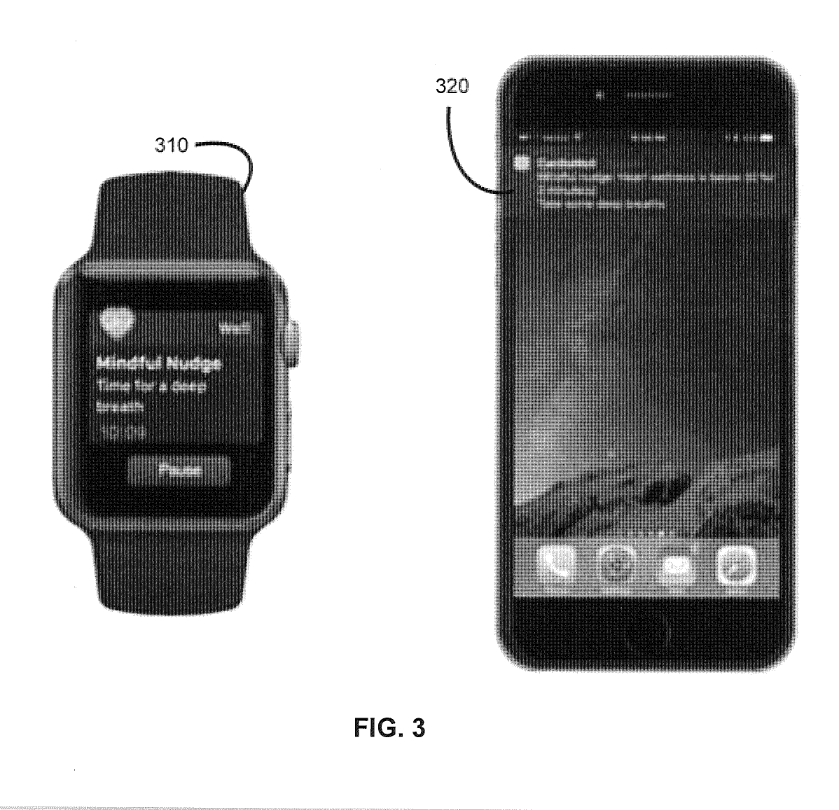

FIG. 3 provides electronic devices including notifications provided by a behavior-modification system in accordance with some embodiments.

As shown in FIG. 3, example smart devices such as smart watch 310 and smart phone 320 can be configured to display biofeedback notifications described herein. When the biofeedback settings for a particular parameter are triggered (e.g., heart rate variability), a message in the form of a notification can appear on the smart watch, the smart phone, or other smart device providing feedback to the user that his/her heart rate variability measure has reached a preconfigured limit. By configuring the notifications for heart rate, heart rate variability, and blood pressure, the user can become notified and reminded to perform, for example, breathing exercises in order to improve heart function and reduce blood pressure. By combining notifications with breathing exercise, the user can better keep his/her blood pressure from becoming high resulting in reduced blood pressure over time.

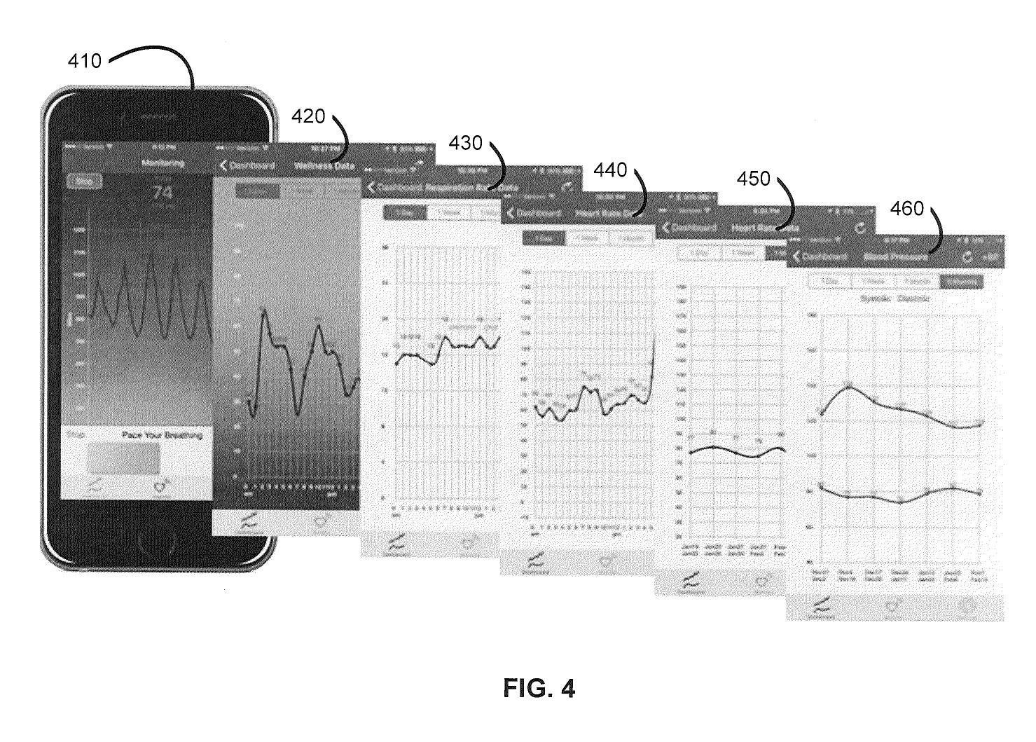

FIG. 4 provides a training module of a behavior-modification system in accordance with some embodiments.

As shown in FIG. 4, the behavior-modification system can include a user interface of an instruction and/or training module 410 in an application installed on a smart phone. Included in the user interface is, for example, a configurable breath pacer designed to help users reach a respiration rate of six breaths per minute. When used with a heart rate monitor, it allows for the display of pulse and heart rate variability data so the user can see how the breathing affects his/her heart rate. The user can be provided with instructions and/or various training exercises to help improve his/her heart rate variability. As shown in wellness data window 420, the user's heart rate variability data can be displayed for over 24 hours of continuous monitoring. Included is the ability to tag a particular event that occurs (e.g., feeling angry) allowing that event to be correlated to one or more physiological readings at the time (e.g., blood pressure, pulse). As shown in respiration rate data window 430, a trend of respiration rate over 24 hours can be displayed. As shown in heart rate data window 440, the results of heart rate data over the past 24 hours can be displayed. As shown in heart rate data window 450, the heart rate data over the past 3 months can be displayed. As shown in blood pressure window 460, blood pressure over the past three months can be displayed.

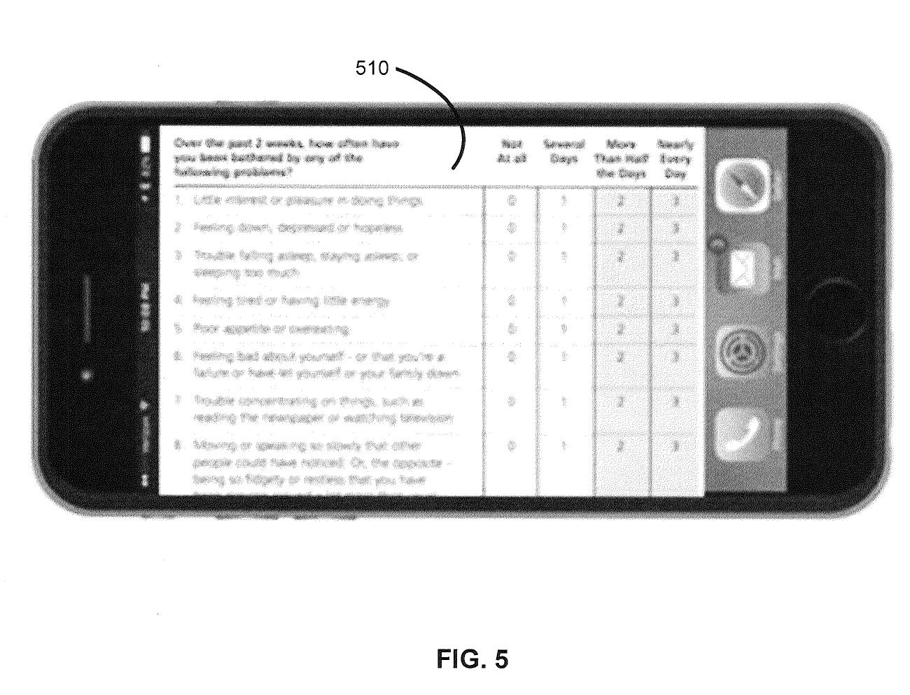

FIG. 5 provides a self-assessment questionnaire of a behavior-modification system in accordance with some embodiments.

As shown in FIG. 5, an example of a self-assessment questionnaire is displayed where the user can takes self-report surveys to help assess their health and mood. One such self-assessment questionnaire can be a depression measurement scale to help assess changes in depression levels. Learning to breathe better and practicing breathing at six breathes per minute is known to reduce depression. Self-assessment questionnaires are tools to help assess changes in depression.

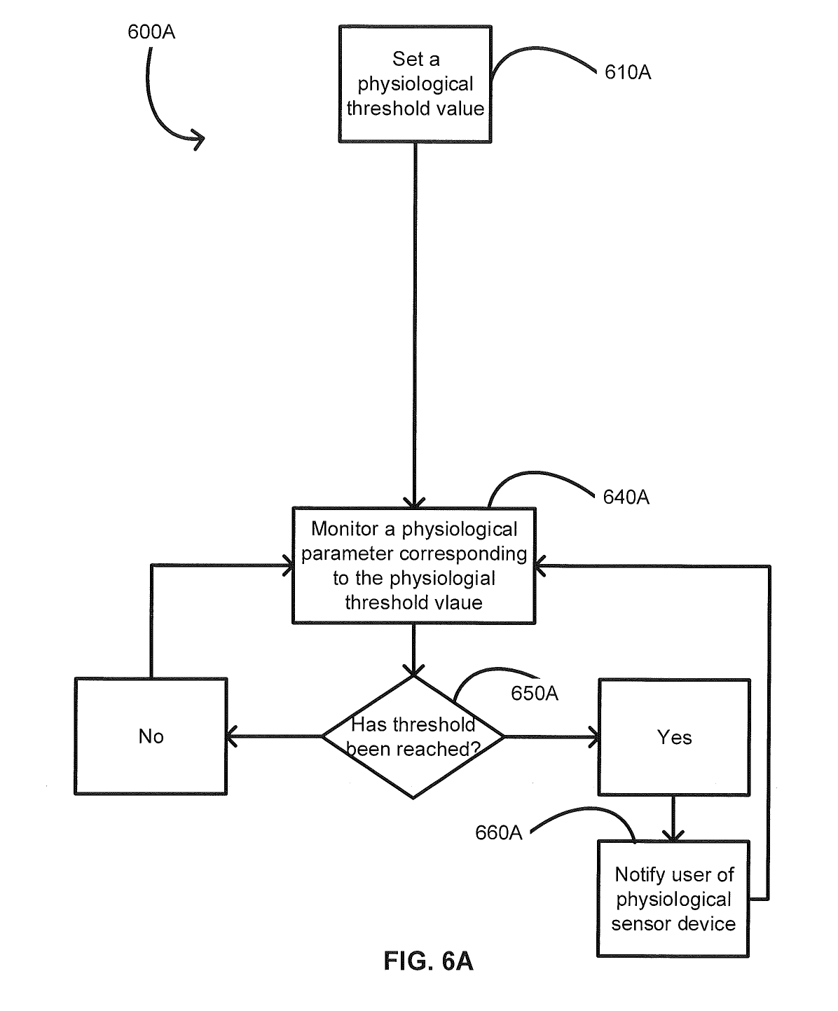

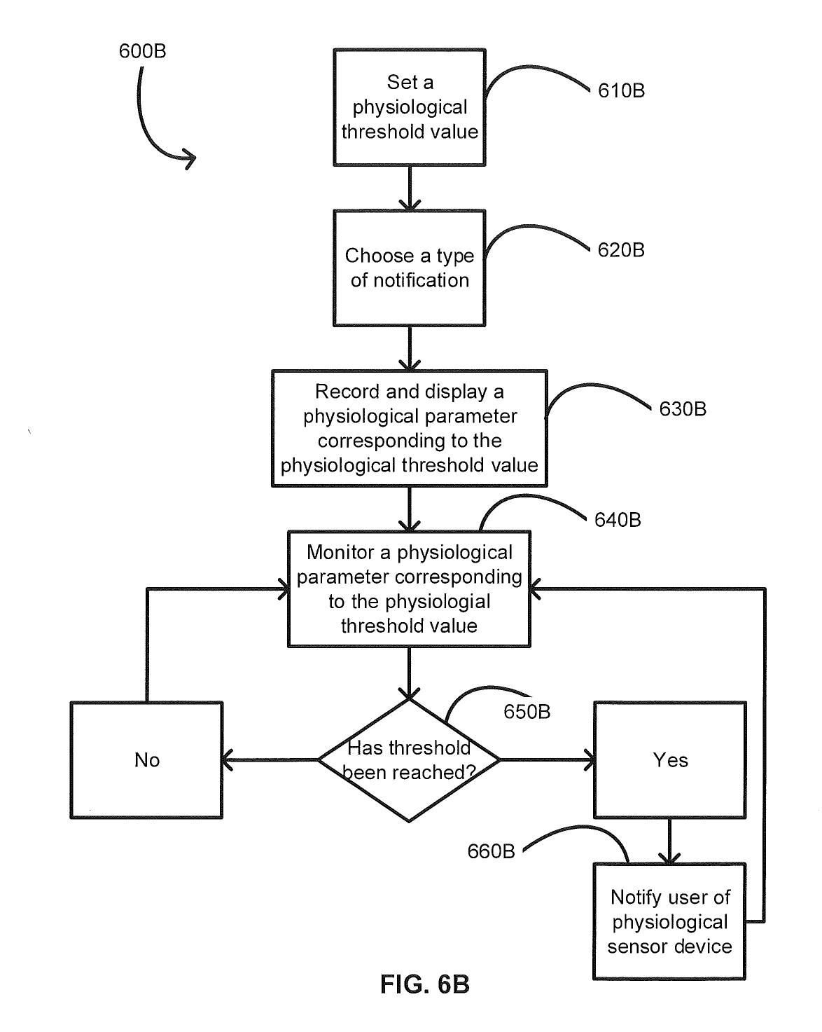

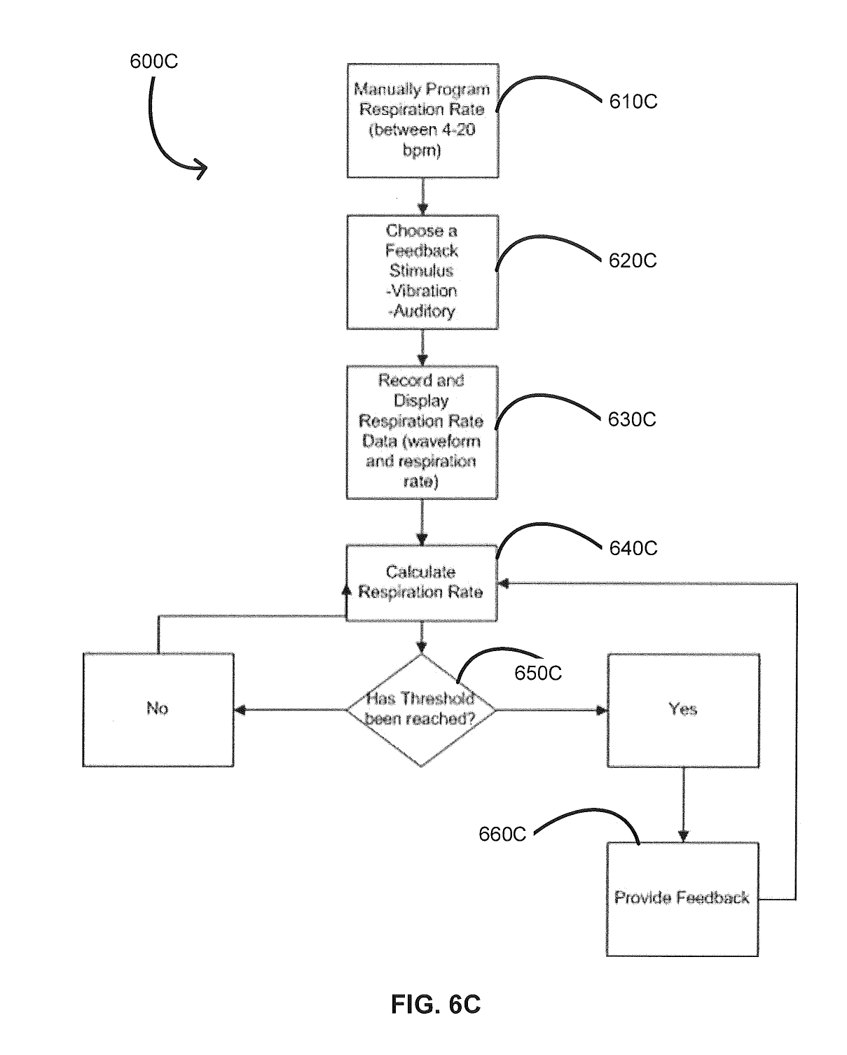

FIG. 6A provides a schematic illustrating a method of a behavior-modification system for providing notifications such as biofeedback notifications in accordance with some embodiments.

As shown in FIG. 6A, the method 600A of the behavior-modification system for providing notifications can include a first step 610A of setting a physiological threshold value such as a heart rate, a respiration rate, or the like. The physiological threshold value can be directly set on a physiological sensor device by a user thereof, or the physiological threshold value can be remotely set on the physiological sensor device by the user or a healthcare professional through a network-connected client computer, for example, in a web browser interface. Upon setting the physiological threshold value, the method can monitor a physiological parameter corresponding to the physiological threshold value in a second step 640A. The method of the behavior-modification system can be configured to periodically determine or determine in real-time if the physiological parameter exceeds the physiological threshold value in a third step 650A. If the physiological parameter does not exceed the physiological threshold value in the third step 650A (e.g., the third step 650A=“No”), the method can continue to monitor the physiological parameter corresponding to the physiological threshold value in the second step 640A. If the physiological parameter does exceed the physiological threshold value in the third step 650A (e.g., the third step 650A=“Yes”), the method can advance to a fourth step 660A, in which case the user of the physiological sensor device can be notified that the physiological parameter exceeded the physiological threshold value. After the fourth step 660A, the method of the behavior-modification system can continue to monitor the physiological parameter corresponding to the physiological threshold value in the second step 640A.

FIG. 6B provides a schematic illustrating a method of a behavior-modification system for providing notifications such as biofeedback notifications in accordance with some embodiments.

As shown in FIG. 6B, the method 600B of the behavior-modification system for providing notifications can include a first step 610B of setting a physiological threshold value such as a heart rate, a respiration rate, or the like. The physiological threshold value can be directly set on a physiological sensor device by a user thereof, or the physiological threshold value can be remotely set on the physiological sensor device by the user or a healthcare professional through a network-connected client computer, for example, in a web browser interface. In a second step 620B, the user (or a healthcare professional) can choose a type of notification (e.g., vibratory notification, auditory notification, visual notification, etc.) should a physiological parameter corresponding to the physiological threshold value exceed the physiological threshold value. Like the first step 610B, the second step 620B can be effected through a network-connected client computer, for example, in a web browser interface. In a third step 630B, the physiological sensor device, a smart device (e.g., smart watch, smart phone, etc.) communicatively coupled thereto, a network-connected client computer, or a combination thereof can record and display the physiological parameter corresponding to the physiological threshold value. In a fourth step 640B, the method can monitor the physiological parameter corresponding to the physiological threshold value. The method of the behavior-modification system can be configured to periodically determine or determine in real-time if the physiological parameter exceeds the physiological threshold value in a fifth step 650B. If the physiological parameter does not exceed the physiological threshold value in the fifth step 650B (e.g., the fifth step 650B=“No”), the method can continue to monitor the physiological parameter corresponding to the physiological threshold value in the fourth step 640B. If the physiological parameter does exceed the physiological threshold value in the fifth step 650B (e.g., the fifth step 650B=“Yes”), the method can advance to a sixth step 660B, in which case the user of the physiological sensor device can be notified that the physiological parameter exceeded the physiological threshold value. After the sixth step 660B, the method of the behavior-modification system can continue to monitor the physiological parameter corresponding to the physiological threshold value in the fourth step 640B.

FIG. 6C provides a schematic illustrating a method of a behavior-modification system for providing notifications such as biofeedback notifications in accordance with some embodiments.

As shown in FIG. 6C, the method 600C of the behavior-modification system can be configured to provide respiration rate biofeedback notifications. In a first step 610C, a threshold value can be set for a respiration rate. The threshold value for the respiration rate can be directly set on a respiration sensor device by a user thereof, or the threshold value for the respiration rate can be remotely set on the respiration sensor device by the user or a healthcare professional through a network-connected client computer, for example, in a web browser interface. In a second step 620C, the user (or a healthcare professional) can choose a type of notification (e.g., vibratory notification, auditory notification, visual notification, etc.) should a respiration rate corresponding to the threshold value for the respiration rate exceed the threshold value for the respiration rate. Like the first step 610C, the second step 620C can be effected through a network-connected client computer, for example, in a web browser interface. In a third step 630C, the respiration sensor device, a smart device (e.g., smart watch, smart phone, etc.) communicatively coupled thereto, a network-connected client computer, or a combination thereof can record and display the respiration rate. In a fourth step 640C, the method can monitor the respiration rate. The method of the behavior-modification system can be configured to periodically determine or determine in real-time if the respiration rate exceeds the threshold value for the respiration rate in a fifth step 650C. If the respiration rate does not exceed the threshold value for the respiration rate in the fifth step 650C (e.g., the fifth step 650C=“No”), the method can continue to monitor the respiration rate in the fourth step 640C. If the respiration rate does exceed the threshold value for the respiration rate in the fifth step 650C (e.g., the fifth step 650C=“Yes”), the method can advance to a sixth step 660C, in which case the user of the respiration sensor device can be notified that the respiration rate exceeded the threshold value for the respiration rate. After the sixth step 660C, the method of the behavior-modification system can continue to monitor the respiration rate in the fourth step 640C.

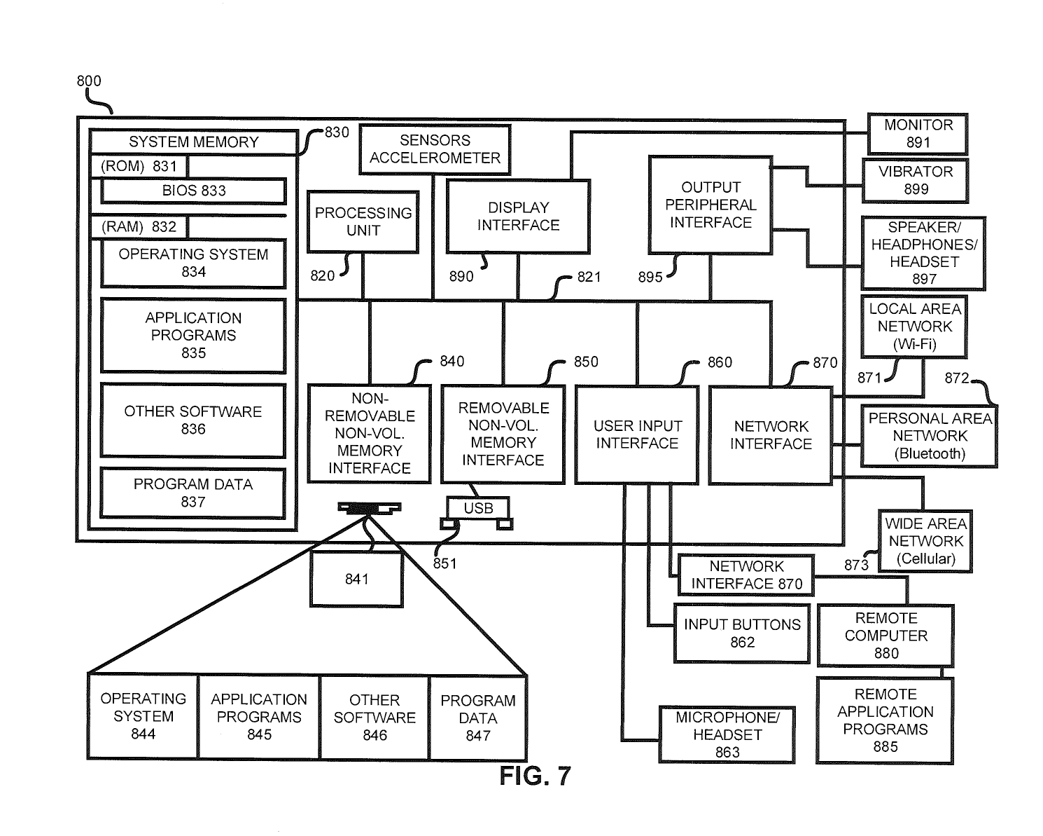

FIG. 7 illustrates a computing system that, in whole or in part, can be part of one or more of the electronic devices (e.g., physiological sensor device, smart device, server computer, client computer, etc.) provided herein. With reference to FIG. 7, components of the computing system 800 may include, but are not limited to, a processing unit 820 having one or more processing cores, a system memory 830, and a system bus 821 that couples various system components including the system memory 830 to the processing unit 820. The system bus 821 may be any of several types of bus structures selected from a memory bus or memory controller, a peripheral bus, and a local bus using any of a variety of bus architectures.

Computing system 800 typically includes a variety of computing machine-readable media. Computing machine-readable media can be any available media that can be accessed by computing system 800 and includes both volatile and nonvolatile media, and removable and non-removable media. By way of example, and not limitation, computing machine-readable media use includes storage of information, such as computer-readable instructions, data structures, other executable software or other data. Computer-storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium which can be used to store the desired information and which can be accessed by the computing device 800. Transitory media such as wireless channels are not included in the machine-readable media. Communication media typically embody computer readable instructions, data structures, other executable software, or other transport mechanism and includes any information delivery media. As an example, some client computing systems on the network 220 of FIG. 7 might not have optical or magnetic storage.

The system memory 830 includes computer storage media in the form of volatile and/or nonvolatile memory such as read only memory (ROM) 831 and random access memory (RAM) 832. A basic input/output system 833 (BIOS) containing the basic routines that help to transfer information between elements within the computing system 800, such as during start-up, is typically stored in ROM 831. RAM 832 typically contains data and/or software that are immediately accessible to and/or presently being operated on by the processing unit 820. By way of example, and not limitation, FIG. 7 illustrates that RAM 832 can include a portion of the operating system 834, application programs 835, other executable software 836, and program data 837.

The computing system 800 can also include other removable/non-removable volatile/nonvolatile computer storage media. By way of example only, FIG. 7 illustrates a solid-state memory 841. Other removable/non-removable, volatile/nonvolatile computer storage media that can be used in the example operating environment include, but are not limited to, USB drives and devices, flash memory cards, solid state RAM, solid state ROM, and the like. The solid-state memory 841 is typically connected to the system bus 821 through a non-removable memory interface such as interface 840, and USB drive 851 is typically connected to the system bus 821 by a removable memory interface, such as interface 850.

The drives and their associated computer storage media discussed above and illustrated in FIG. 7, provide storage of computer readable instructions, data structures, other executable software and other data for the computing system 800. In FIG. 7, for example, the solid state memory 841 is illustrated for storing operating system 844, application programs 845, other executable software 846, and program data 847. Note that these components can either be the same as or different from operating system 834, application programs 835, other executable software 836, and program data 837. Operating system 844, application programs 845, other executable software 846, and program data 847 are given different numbers here to illustrate that, at a minimum, they are different copies.

A user may enter commands and information into the computing system 800 through input devices such as a keyboard, touchscreen, or software or hardware input buttons 862, a microphone 863, a pointing device and/or scrolling input component, such as a mouse, trackball or touch pad. The microphone 863 can cooperate with speech recognition software. These and other input devices are often connected to the processing unit 820 through a user input interface 860 that is coupled to the system bus 821, but can be connected by other interface and bus structures, such as a parallel port, game port, or a universal serial bus (USB). A display monitor 891 or other type of display screen device is also connected to the system bus 821 via an interface, such as a display interface 890. In addition to the monitor 891, computing devices may also include other peripheral output devices such as speakers 897, a vibrator 899, and other output devices, which may be connected through an output peripheral interface 895.

The computing system 800 can operate in a networked environment using logical connections to one or more remote computers/client devices, such as a remote computing system 880. The remote computing system 880 can a personal computer, a hand-held device, a server, a router, a network PC, a peer device or other common network node, and typically includes many or all of the elements described above relative to the computing system 800. The logical connections depicted in FIG. 7 can include a personal area network (PAN) 872 (e.g., Bluetooth®), a local area network (LAN) 871 (e.g., Wi-Fi), and a wide area network (WAN) 873 (e.g., cellular network), but may also include other networks such as a personal area network (e.g., Bluetooth®). Such networking environments are commonplace in offices, enterprise-wide computer networks, intranets and the Internet. A browser application may be resident on the computing device and stored in the memory.

When used in a LAN networking environment, the computing system 800 is connected to the LAN 871 through a network interface or adapter 870, which can be, for example, a Bluetooth® or Wi-Fi adapter. When used in a WAN networking environment (e.g., Internet), the computing system 800 typically includes some means for establishing communications over the WAN 873. With respect to mobile telecommunication technologies, for example, a radio interface, which can be internal or external, can be connected to the system bus 821 via the network interface 870, or other appropriate mechanism. In a networked environment, other software depicted relative to the computing system 800, or portions thereof, may be stored in the remote memory storage device. By way of example, and not limitation, FIG. 7 illustrates remote application programs 885 as residing on remote computing device 880. It will be appreciated that the network connections shown are examples and other means of establishing a communications link between the computing devices may be used.

As discussed, the computing system 800 can include a processor 820, a memory (e.g., ROM 831, RAM 832, etc.), a built in battery to power the computing device, an AC power input to charge the battery, a display screen, a built-in Wi-Fi circuitry to wirelessly communicate with a remote computing device connected to network.

It should be noted that the present design can be carried out on a computing system such as that described with respect to FIG. 7. However, the present design can be carried out on a server, a computing device devoted to message handling, or on a distributed system in which different portions of the present design are carried out on different parts of the distributed computing system.

Another device that may be coupled to bus 821 is a power supply such as a DC power supply (e.g., battery) or an AC adapter circuit. As discussed above, the DC power supply may be a battery, a fuel cell, or similar DC power source that needs to be recharged on a periodic basis. A wireless communication module can employ a Wireless Application Protocol to establish a wireless communication channel. The wireless communication module can implement a wireless networking standard.

In some embodiments, software used to facilitate algorithms discussed herein can be embodied onto a non-transitory machine-readable medium. A machine-readable medium includes any mechanism that stores information in a form readable by a machine (e.g., a computer). For example, a non-transitory machine-readable medium can include read only memory (ROM); random access memory (RAM); magnetic disk storage media; optical storage media; flash memory devices; Digital Versatile Disc (DVD's), EPROMs, EEPROMs, FLASH memory, magnetic or optical cards, or any type of media suitable for storing electronic instructions.

Note, an application described herein includes but is not limited to software applications, mobile apps, and programs that are part of an operating system application. Some portions of this description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. These algorithms can be written in a number of different software programming languages such as C, C+, or other similar languages. Also, an algorithm can be implemented with lines of code in software, configured logic gates in software, or a combination of both. In an embodiment, the logic consists of electronic circuits that follow the rules of Boolean Logic, software that contain patterns of instructions, or any combination of both.

Having described some embodiments and various features thereof of the systems and methods provided herein, additional embodiments and various features of those additional embodiments will now be described. Again, it should also be understood that a particular embodiment provided anywhere herein can have features that can be readily separated from the particular embodiment and optionally combined with or substituted for features of any of a number of other embodiments provided herein.

Systems and methods provided herein can be used to treat and manage hypertension and improve wellness. Clinical research shows diaphragmatic breathing can reduce sympathetic nervous system activity and increases the bodies' ability to control blood pressure. In some embodiments, systems and methods provided herein apply clinical research into a user friendly application running on mobile devices that are supported by physiological sensors to help a user lower blood pressure.

There is no current solution that leverages a smart watch or other smart device to monitor and train users to improve heart rate variability, respiratory sinus arrhythmia, and breathing throughout their day in order to reduce and control blood pressure and depression.

Advantages of the systems and methods provided herein include, in no particular order, a) removing dependence from conventional biofeedback devices for breathing and heart rate variability that run exclusively on personal computers; b) removing requirements for the user to be situated at a desk, enabling the user to become aware of how their experience/situation affects their physiology through feedback throughout the day; c) removing dependence on required skilled technicians for the conventional biofeedback devices; d) continuous monitoring and feedback while the user is ambulatory throughout the day, whereas conventional biofeedback devices do not provide such continuous monitoring; e) employing wireless, wearable, and mobile technologies, as well as one or more sensors for ECG, HRV, RSA feedback and user-individualized methods based on sensor data from the foregoing sensors; and notifying users to practice breathing throughout the day.

A purpose of the systems and methods provided herein is to improve health, wellness, blood pressure and depression. A mobile blood pressure management and control solution that guides users through breathing exercises and makes them aware of instances when their heart rate variability is low and under physiological stress allowing them awareness and ability to better control their breathing. It integrates with physiological sensors that detect heart rate, heart rate variability (HRV), respiration and blood pressure to allow for quick self-learning, remote monitoring and management by healthcare professionals. Systems and methods provided herein assist the user to manage stressful situations to prevent increases in blood pressure. Physiological and situational awareness along with guided breathing exercises allow the user to stabilize blood pressure and may reduce dependence on medication over time.

By using smart devices such as watches, phones and tablets connected to physiological monitoring sensors, patients become aware of their physiological state and learn skills to manage their physiology and emotions. The uniqueness of the solution provided herein is the way it allows a user to gain insight into his/her physiological reactions to stress and the “nudges” in the applications on smart devices to make behavioral changes resulting in lowered blood pressure.

Systems and methods provided herein generally related to a digital therapeutic biofeedback communication system suitable for adults and children. It is composed of a system and method allowing for breathing and heart rate variability training to improve a user's blood pressure. Its principle use is physiological feedback of autonomic nervous system function to allow for physiological auto-regulation. Systems and methods provided herein can be understood as a digital therapeutic to better manage hypertension.

In some embodiments, the system comprises a “smart watch” worn by an individual that communicates with integrated physiological sensors allowing the user to receive feedback and instruction at times when heart rate variability (HRV) decreases, pulse rate increases, respiration increases, and increases in other physiological measurements.

Methods comprise breathing and heart rate variability training programs with reminders, notifications (nudges) that allows the user to reduce blood pressure over time.

The data received from sensors will display on the smart watch allowing the user to view their heart rate variability and breathing patterns and take action to improve their condition.

People are living longer and healthcare require people to better care for themselves. Patients are taking greater control and responsibility for their health. Medications are the main form of treatment and come with unwanted side effects. Lifestyle changes and management of stress are major contributors to poor health. Stress management together with lifestyle modifications and a supervised medical regimen is important to control blood pressure and prevent a stroke.

Managing blood pressure is especially important for patients who already had a stroke or heart attack. Studies show that every 10 mmHg reduction in systolic blood pressure reduce the risk of heart attack by about a fifth, stroke and heart failure by about a quarter, and the risk of death from any cause by about 13 percent. Current guidelines have moved recommendation from 130/90 to 120/80.

This solution provides a way for users to naturally improve their lifestyle and reduce their stress while improving their breathing to help lower stress, blood pressure, and depression while allowing them to become less dependent on medications.

Many diseases are a result of, or are precipitated and influenced by, stress. Research shows biofeedback and auto-regulation methods such as deep breathing help manage disease conditions such as hypertension, depression, anxiety, and pain. The difficulty in the current practice lies in providing relevant real-time feedback to the user when in a stressful condition that contains relevance to their physiology so they can do something about it in the moment.

Systems and methods provided herein allow users to receive feedback of their respiration, respiratory sinus arrhythmia (RSA), heart rate, heart rate variability (HRV), blood pressure and stress response using a smart device such as a smart watch or smart phone as the primary source of processing and feedback. A unique method run by software on the smart device trains the user to control heart rate, heart rate variability, and respiration. Over time this results in lowering blood pressure and depression.

In some embodiments, a PBS (personal biofeedback system) can be worn by consumers to improve health. The PBS can be made up of a smart device, an application, and one or more physiological monitoring sensors (PMS).

In some embodiments, a system is provided with a method for physiological auto-regulation using biofeedback and management of hypertension and depression.

A goal of the system is to change behavior and improve health.

It is a purpose to describe components of the system that are made up of wearable and non-wearable physiological sensors wirelessly or otherwise connected to a smart watch, tablet, and/or smart phone (smart device).

An additional purpose of the systems and methods provided herein is to provide feedback, instruction, and training to the user in order for the user to change his/her physiological state.

In some embodiments, a system comprises a smart watch, an application running on the smart watch, and integrated physiological sensors in the smart watch.

In some embodiments, a method provided herein comprises the following components within the application: biofeedback notifications; instructions and training; event tagging; and self-assessment questionnaires.

In some embodiments, a personal biofeedback system (PBS) can be worn by consumers to improve health. The PBS is made up of a smart watch and/or smart phone and/or tablet (all of which are considered smart devices herein), an application running one or more of the smart devices, and one or more physiological monitoring sensors (PMS). The smart watch and/or smart phone can be configured to receive real-time physiological data from the physiological sensors and process the data for display to the user in a meaningful way. It can process and display data using preprogrammed threshold values that provide a variety of feedback stimuli and instruction. Feedback stimuli can be in the form of visual, sound, audible instruction, or tactile vibration from the watch or phone.

The system is made up of hardware and software. The hardware includes, for example, one or more smart devices and one or more connected physiological sensors. The software includes applications that can be executed by a processor on one or more of the smart devices or a computer. The software also includes a method of feedback and instruction to the user based on his/her physiological activity.

The smart devices can provide feedback to the user through supported applications. The purpose of the feedback is to increase awareness of physiological activity and allow for physiological auto-regulation. Methods can be deployed to allow for physiological auto-regulation of and management of hypertension and depression, as well as congestive heart failure, shortness of breath, angina, and other related cardiorespiratory diseases.

In some embodiments, the system can be made up of off-the-shelf smart devices that process and display signals from built-in or external physiological sensors. A visual display of the signals in conjunction with or without tactile and auditory feedback provides awareness to the user.

The system benefits from a low-cost, low-power mobile computing device and takes advantage of off-the-shelf smart devices and operating systems.

A smart watch is considered to be a device like the Apple Watch, Android Wear smart watch, or a similar type wrist-worn device that has integrated an CPU and runs an operating system. The smart watch software can be programmed in such a way as to provide the most appropriate type of feedback and instruction to the user at the right time.

In some embodiments, the smart watch can be configured to run either an iOS or Android operating system. The software applications can be installed by the user on the smart watch.

In some embodiments, the smart watch can support long and short-range wireless protocols such as cellular, Bluetooth® and Wi-Fi.

In some embodiments, one or more physiological sensors can be integrated into the smart watch such as heart rate, ECG, oximeter, blood pressure device, electromyography (emg), and electrical conductance and processed such that one or more calculations of heart rate, heart rate variability, stress level, tension, and respiratory sinus arrhythmia (RSA) can be displayed to the user.

Alternatively a smart device like an smart phone or tablet can be used by itself or in conjunction with a smart watch. When used with a smart watch, the smart phone can act as a hub allowing the smart watch to send physiological data to a display while simultaneously sending the data to one or more database servers located on the Internet.

The smart devices can process the signals sent from the sensors and display data along with preprogrammed feedback. Feedback can be in the form of visual, sound, audible instruction, or tactile vibration from the smart device.

In some embodiments, the data received by the smart device can be immediately displayed for viewing and feedback. The smart device can be connected to the Internet through a wireless network such as a wireless cellular network, Wi-Fi, Bluetooth®, or other.

Physiological sensors can include one or more sensors selected from an integrated heart rate sensor, a temperature sensor, a pulse oximetry sensor, a galvanic skin response sensor, a blood pressure sensor, and other physiological sensors, as well as activity type sensors such as a gyroscope.

Alternatively the sensors can be external to the smart watch. When external to the smart watch, the smart watch and/or smart phone receives signals from the external sensors and processes the data for display to the user in a meaningful way.

External physiological sensors can include those built into garments such as a shirt, shorts, pants, a brazier, a jacket, a vest, or some other article of clothing.

External sensors can be a smart garment worn by the user that is laden with a variety of physiological sensors such as ECG and respiration sensors. External sensors can include stand-alone devices including one or more sensors selected form blood pressure sensors, respiration sensors, EEG sensors, any one or more of which can be configured to communicate wirelessly to the smart device.

External sensors can include a blood pressure device that is connected to a data base via a cellular (GSM, CDMA), Wi-Fi, or Bluetooth® Internet connection.

The wearable sensors can communicate to the smart device using one or more different kinds of personal area network (PAN) protocols selected from Qualcom, Zig-Bee, Bluetooth®, and MIMO; however such PAN protocols are not limited to the foregoing.

Data transmitted by wearable physiological sensors and received directly by the smart device can be subsequently transmitted and stored in real-time over the Internet, such that the data can be retrieved and visualized from a web browser or other software application.

The data can be viewed locally on the smart device in real-time for patient monitoring, or the data can be stored and visualized in the form of graphs and trends to allow the user to self-monitor their progress while simultaneously allowing the data to be shared with healthcare professionals.

The system can include use of a connected blood pressure monitor that transmits blood pressure by way of Bluetooth®, cellular, or Wi-Fi to a database located on the Internet for storage. Data can be transmitted from the blood pressure device through a cellular chip, gateway, hub, router, or directly through the smart device. The blood pressure data can be made available to the user and to remote health care providers for viewing.

Separately, the user can be provided with a weight scale, a blood glucose monitor, a smart medication dispensary device, or some other kind of physiological monitoring device that communicates either directly to the smart device or directly to a database through a cellular, Wi-Fi, or other wireless Internet connection.

An application running on a smart device can be programmed directly or indirectly from a native application or from an application used by a healthcare provider allowing the provider to remotely monitor the user and manage their health.

Important to the solution in some embodiments is the method that includes use of a connected blood pressure monitor that transmits blood pressure by way of Bluetooth®, cellular, or Wi-Fi to a database located in the Internet for storage. Data can be transmitted from the blood pressure device through a gateway, hub, router, or directly through the smart watch or smart phone. The blood pressure data can be made available to the user and to remote health care providers for viewing.

The application can run on a smart device, which provides a mechanism to employ the methods provided herein.

In some embodiments, methods comprise biofeedback notifications; instruction and training; event tagging; and self-assessment questionnaires.

Biofeedback notifications can be programmed directly on a smart device or remotely from a web browser portal by the user or by a healthcare professional. The application and web browser portal allows for a variety of programmed criteria and feedback algorithms to allow for immediate feedback based on any one or more of the following criteria:

(1) Respiratory criteria data such as breathing too fast; breathing too slow; breath holding; breathing too deep; breathing too shallow; irregular breathing; and sighing.

(2) Heart rate variability criteria data such as heart rate too fast; heart rate too slow; no heart rate; irregular heart rate; low, moderate, or high HRV; and rMSSD HRV.

(3) RSA criteria data such as high, low, moderate RSA; and coherence.

(4) Blood pressure criteria data such as high or low systolic blood pressure; and high or low diastolic blood pressure.

The biofeedback notifications function to alert the user that a particular physiological signal has reached criteria. The goal of the feedback is to modify behavior so as to bring the physiological signal to a desired level. The principles of modifying the behavior are based on positive and negative reinforcement and take root in classical conditioning.

Feedback includes programmed thresholds set on the smart device that trigger visual, audio, or tactile feedback in the form of audio, tactile, and visual nudges, reminders, and alerts. The immediate feedback cues the user to their physiological activity as displayed on a smart device (e.g., fast and shallow breathing, fast and/or normal heart rate). The feedback increases situational awareness of events that cause physiological arousal and increased blood pressure.

Users are provided with various forms of feedback to help promote healthy breathing, RSA, and HRV. For example, one threshold condition includes feedback to help the user recognize that he/she is breathing too fast (e.g., respiration rate>16 breaths per min [bpm]). Another example includes providing feedback when the user's rMSSD HRV becomes too low. The feedback can be, in some cases, followed by instructions to help the user bring their respiration into a more normal rate.

The biofeedback notification can be used to notify the user when he/she is not breathing optimally as a form of negative reinforcement, or to let a user know that he/she is breathing well as a form of positive reinforcement

Auditory feedback to the user can include music, sounds, and instructions. In some embodiments, the user can choose and add various music and sound files to be used as feedback.

Additionally, instructional feedback can be used such that the user is instructed on breathing at a desired respiratory rate and cycle.

The smart device software is programmed in such a way as to provide the most appropriate type of feedback and instruction to the user at the right time.

Typically, the data received by the smart device is immediately displayed for viewing and feedback. As an option, the data is stored into memory.

In some embodiments, biofeedback notifications are used to instruct and train a user in diaphragmatic breathing with or without the use of a physiological monitoring device by allowing the user to receive feedback about their respiration and heart rate activity.

The instruction and training program can teach the user to improve his/her breathing patterns resulting in a reduction in blood pressure and depression over time.

The instruction and training method can include paced breathing exercises. Included in the paced breathing exercises is a configurable breathing rate and depth pacer. The user can be instructed to breath at a rate that mimics the auto-generated respiratory rate breathing pacer while receiving real-time physiological feedback from corresponding physiological monitoring sensors worn.

The breathing pacer feature can train the user to breathe at varying respiration rates as needed to train the user to breathe for a certain health benefit. This allows the user to receive feedback and instruction that will allow for the voluntary change in respiratory rate. The goal is to change involuntary breathing patterns over time.

A goal is to instruct users to breathe at about six breaths per minute.

Training session lengths can vary from 30 seconds to 60 minutes or longer.

A training session, for example, can include heart rate and respiratory feedback with periodic deep breathing exercises interspaced throughout the day.

Another training session can be, for example, a one-minute guided mindfulness meditation while breathing at about six breaths per minute.

A component of the solution is to share the information with healthcare professionals who are responsible for ensuring the health and wellness of the consumer/user.

A view into the data for the healthcare provider allows the healthcare provider to see changes in weight, heart rate, blood pressure, respiration rate, HRV, and/or other health measures, as well as create thresholds and goals that allow for the improvement of the user's health. One of the goals of the solution is to help the patient manage blood pressure. By providing blood pressure related data to healthcare professionals, the healthcare organization can utilize tools to help the user reduce blood pressure and other related medications as the users health improves.

Data views include trends and graphs allowing for the healthcare providers to see trends in the health of the user.

The solution can help healthcare organizations not only improve the health of their customers but also reduce visits to the emergency room and clinics by allowing the healthcare organization to communicate with the patients remotely and ensure their medical condition is under control.

In some embodiments, the user can allow other users permission to receive his/her physiological data. In this case, both users can keep track of each other's progress.

To support the reduction of blood pressure, the system allows for the provision and triggering of medication reminders and questionnaires to assess mood, behavior, events, and activities.

An important chronic disease that systems and methods provided herein aim to manage is hypertension and depression. Systems and methods can be incorporated for the treatment and management of diabetes, anxiety, pain, asthma, emphysema, panic attacks, sleep apnea, phobias, paralysis, coma, CHF, obesity, ADD/ADHD, and other diseases.

The method includes event tagging and self-assessment questionnaires so as to associate physiological responses to an event and assess a user's mood and feelings.

Event tagging acts as a kind of diary and symptom inventory allowing the user to keep track of events and symptoms.

Self-assessment questionnaires can include the Becks Depression Inventory (BDI) or similar psychometric tools to help assess the users' mood, feelings, beliefs, or opinion.

As such, provided herein in some embodiments are systems and/or methods including any one or more of the following features:
a blood pressure-management apparatus comprising a smart device and physiological sensors that provide feedback, training, and the ability to manage medication, tag events and complete questionnaires
a smart watch, smart phone, or tablet and physiological monitoring sensors
user-management of his/her blood pressure
biofeedback notifications, instructions and training, medication management, event tagging, and/or self-assessment questionnaires
treatment of depression, anger, angina, anxiety, stress-related disorders, asthma, emphysema, panic attacks, sleep apnea, and phobias
modification of a user's behavior
smart watch, smart phone, or tablet displays physiological data and provides feedback to the user
feedback in the form of tactile, auditory, or visual feedback
the physiological monitoring sensors are wearable and wirelessly communicate with the smart watch, smart phone or tablet (herein referred to as smart devices)
the physiological sensors can be integrated into the smart device
the sensors are noninvasive and worn either wirelessly and wired within garments and accessories such as wrist bands, watches, chest straps, rings, t-shirts, underwear and bra, shoes and slippers, wetsuits, headbands, hats and other accessories such as belts and vests, that are worn by the user and communicates wirelessly to a smart device
fastened to the user either by adhesive, a strap, or a belt
the physiological sensors include electroencephalograms (EEG), electromyography (EMG), electrocardiography (ECG), heart rate variability (HRV), respiratory sinus arrhythmia (RSA), respiration, blood pressure, temperature, peripheral saturation of oxygen (SpO2), weight, and skin conductance/resistance
a sensor such as an accelerometers and gyrometer collects data and provides feedback to the user on caloric burn, distance traveled, and body position
an ECG sensor
the sensor also includes heart rate variability calculations
a wearable wireless ECG sensor
the ECG sensor is integrated in a smart watch
ECG sensors integrated into articles of clothing
the ECG sensors at connected by an adhesive patch directly to the skin
the ECG sensors are connected to the body by use of a strap
the ECG sensor automatically detects heart rate variability
the ECG signal is sent to a smart device for calculation of heart rate variability
the ECG signal can be used to provide feedback, remote monitoring, alerts and further analysis
wireless data transmission by short- and/or long-range wireless protocols
Bluetooth® and/or ZigBee
Wi-Fi, Wi-Fi Max, and/or cellular
a blood pressure monitoring device
the blood pressure monitoring device communicates with the Internet using cellular, LAN, Bluetooth®, and/or Wi-Fi
the data can be used to provide feedback to the user
worn by the user
a stationary stand-alone device
measures pulse
the data is made available to remote health care providers
the data is used to monitor hypertension
the data is used to make medical decisions such as medication management
the data can be used to prevent degradations in health and wellness
a weight scale
the weight scale communicates with the Internet using cellular, LAN, Bluetooth®, and/or Wi-Fi
the data can be used to monitor weight gain
the data can be used to manage hypertension and congestive heart failure
the data is used to make medical decisions
a wearable wireless respiration sensor
the respiration sensor measures respiration rate and depth of breath
the wearable and wireless EEG sensors allow for the monitoring and treatment of seizures, sleep, alertness, awareness, anxiety, depression, coma, and/or peak performance
the EEG sensors can be used to remotely control robotic apparatuses, turn lights on and/or off, or perform a remotely facilitated command
the EEG sensors can be used to improve attention, affect, peak performance, mood, mental and physical condition
the wearable wireless EMG sensors allows for the monitoring and management of stress, muscle tension, and pain
used in the workplace or home to manage TMJ, repetitive-strain injury (RSI), neck and shoulder pain, and/or headaches
biofeedback notifications programmed to provide user feedback
biofeedback notifications based on HRV and/or rMSSD calculations
biofeedback notifications configured to notify the user that his/her HRV or rMSSD calculations have reached a predefined threshold
predefined thresholds are programed by the user on the remote device, automatically, or remotely by a health care provider
biofeedback notifications are based on weight, pulse, blood pressure, temperature, skin conductance, respiration depth and respiration rate
instructions on how diaphragmatic breathing
learning to breathe at the user's resonant frequency
trying to breathe at about six breathes per minute
training sessions enabling the user to practice breathing at various breathing depths and rates
enabling the user to practice breathing at about six breathes per minute
medication management including the ability to monitor medication adherence
reminders for the user to take their medication
recommended changes in medications
Enabling health care professionals to order medication changes
event tagging and self-assessment questionnaires
event tagging includes the ability for a user to associate an event to period in time
the period in time is used to correlate with physiological measurements
physiological measurements can be correlated with events
ability for the user to take short and long psychometric questionnaires
the ability to assess depression, happiness, anxiety, emotions and other psychological states
medication management

Also provided herein in some embodiments is a method, comprising setting a physiological threshold value in a user interface for a user behavior-modification system, wherein at least a portion of the user behavior-modification system is resident in a memory of a first network-connected electronic device configured with a physiological sensor, and wherein the physiological sensor is configured to physiologically couple to the user for monitoring a physiological parameter of the user; monitoring the physiological parameter corresponding to the physiological threshold value to alert the user when the user exceeds the physiological threshold value; and notifying the user with a notification when the user exceeds the physiological threshold value in an event, thereby enabling the user to modify behavior to bring the physiological parameter below the physiological threshold value, wherein the notification is provided through a vibrator, a speaker, or a display screen of the first electronic device or a second network-connected electronic device. In some embodiments, at least a portion of the user behavior-modification system is resident in a memory of a network-connected server computer and a memory of a network-connected client computer, and the server computer is configured to mediate network communication between a) the client computer and the first electronic device or b) the client computer and the second electronic device such that the user can effect setting the physiological threshold value in the behavior-modification system through an electronic-device user interface or a client-computer user interface. In some embodiments, at least a portion of the user behavior-modification system is resident in a memory of a network-connected server computer and a memory of a network-connected client computer, and the server computer is configured to mediate network communication between a) the client computer and the first electronic device or b) the client computer and the second electronic device such that a healthcare professional can effect setting the physiological threshold value for the user in the behavior-modification system. In some embodiments, the method further comprises providing instructions to the user on how to bring the physiological parameter below the physiological threshold value, wherein the instructions are packaged with the notification or provided subsequent to the notification upon a user request for the instructions or a user confirmation from a prompt for the instructions. In some embodiments, the method further comprises providing a training plan to the user on how to maintain the physiological parameter below the physiological threshold value, wherein the training plan is packaged with the notification or provided subsequent to the notification upon a user request for the training plan or a user confirmation from a prompt for the training plan. In some embodiments, the method further comprises providing a training module to the user in accordance with the training plan to aid the user in training to maintain the physiological parameter below the physiological threshold value, wherein the training modules include at last one chart for the physiological parameter vs. time to aid the user in visualizing training effects on the physiological parameter. In some embodiments, the method further comprises providing a self-assessment questionnaire to the user; and providing instructions to the user on how to bring the physiological parameter below the physiological threshold value based upon answers provided by the user on the self-assessment questionnaire. In some embodiments, the behavior-modification system includes tagging, thereby enabling the user to tag the event in which the user exceeds the physiological threshold value with contextual information for the event for ongoing monitoring. In some embodiments, the first electronic device is a physiological sensor device configured with a sensor selected from the group consisting of an electroencephalogram (EEG) sensor, electromyography (EMG) sensor, an electrocardiography (ECG) sensor, a heart rate variability (HRV) sensor, a respiratory sinus arrhythmia (RSA) sensor, a respiration sensor, a blood pressure sensor, a temperature sensor, a peripheral saturation of oxygen (SpO₂) sensor, weight sensor, and a skin conductance/resistance sensor; the second electronic device is communicatively coupled to the first electronic device by a personal area network protocol; and the second electronic device is a smart watch, smart phone, or tablet configured with the physiological sensor. In some embodiments, the first electronic device is a smart watch, smart phone, or tablet configured with the physiological sensor. In some embodiments, the behavior-modification system is used to treat an indication selected from depression, anger, angina, anxiety, a stress-related disorder, asthma, emphysema, panic attacks, sleep apnea, and phobias.

Also provided herein in some embodiments is a method, comprising setting a physiological threshold value in a user interface for a user behavior-modification system, wherein at least a portion of the user behavior-modification system is resident in a memory of a first network-connected electronic device, a memory of a second network-connected electronic device, a memory of a network-connected server computer, and a memory of a network-connected client computer, wherein the server computer is configured to mediate network communication between a) the client computer and the first electronic device or b) the client computer and the second electronic device such that the user can effect setting the physiological threshold value in the behavior-modification system through an electronic-device user interface and a healthcare professional can effect setting the physiological threshold value in the behavior-modification system through a client-computer user interface, and wherein the first electronic device is configured with a physiological sensor for physiologically coupling to the user for monitoring a physiological parameter of the user; monitoring the physiological parameter corresponding to the physiological threshold value to alert the user when the user exceeds the physiological threshold value; notifying the user with a notification when the user exceeds the physiological threshold value in an event, thereby enabling the user to modify behavior to bring the physiological parameter below the physiological threshold value, wherein the notification is provided through a vibrator, a speaker, or a display screen of the second electronic device; and providing instructions to the user on how to bring the physiological parameter below the physiological threshold value, wherein the instructions are packaged with the notification or provided subsequent to the notification upon a user request for the instructions or a user confirmation from a prompt for the instructions.

Also provided herein in some embodiments is a non-transient computer-readable medium containing instructions that when executed by a first network-connected electronic device cause the first electronic device to perform a method, comprising setting a physiological threshold value in a user interface for a user behavior-modification system, wherein at least a portion of the user behavior-modification system is resident in a memory of the first electronic device configured with a physiological sensor, and wherein the physiological sensor is configured to physiologically couple to the user for monitoring a physiological parameter of the user; monitoring the physiological parameter corresponding to the physiological threshold value to alert the user when the user exceeds the physiological threshold value; and notifying the user with a notification when the user exceeds the physiological threshold value in an event, thereby enabling the user to modify behavior to bring the physiological parameter below the physiological threshold value, wherein the notification is provided through a vibrator, a speaker, or a display screen of the first electronic device or a second network-connected electronic device. In some embodiments, at least a portion of the user behavior-modification system is resident in a memory of a network-connected server computer and a memory of a network-connected client computer, and the server computer is configured to mediate network communication between a) the client computer and the first electronic device or b) the client computer and the second electronic device such that the user can effect setting the physiological threshold value in the behavior-modification system through an electronic-device user interface and a healthcare professional can effect setting the physiological threshold value in the behavior-modification system through a client-computer user interface. In some embodiments, the non-transient computer-readable medium, further comprises providing instructions to the user on how to bring the physiological parameter below the physiological threshold value, wherein the instructions are packaged with the notification or provided subsequent to the notification upon a user request for the instructions or a user confirmation from a prompt for the instructions. In some embodiments, the non-transient computer-readable medium further comprises providing a training plan to the user on how to maintain the physiological parameter below the physiological threshold value, wherein the training plan is packaged with the notification or provided subsequent to the notification upon a user request for the training plan or a user confirmation from a prompt for the training plan. In some embodiments, the non-transient computer-readable medium, further comprises providing a training module to the user in accordance with the training plan to aid the user in training to maintain the physiological parameter below the physiological threshold value, wherein the training modules include at last one chart for the physiological parameter vs. time to aid the user in visualizing training effects on the physiological parameter. In some embodiments, the non-transient computer-readable medium further comprises providing a self-assessment questionnaire to the user; and providing instructions to the user on how to bring the physiological parameter below the physiological threshold value based upon answers provided by the user on the self-assessment questionnaire. In some embodiments, the behavior-modification system includes tagging, thereby enabling the user to tag the event in which the user exceeds the physiological threshold value with contextual information for the event for ongoing monitoring. In some embodiments, the first electronic device is a physiological sensor device configured with a sensor selected from the group consisting of an electroencephalogram (EEG) sensor, electromyography (EMG) sensor, an electrocardiography (ECG) sensor, a heart rate variability (HRV) sensor, a respiratory sinus arrhythmia (RSA) sensor, a respiration sensor, a blood pressure sensor, a temperature sensor, a peripheral saturation of oxygen (SpO₂) sensor, weight sensor, and a skin conductance/resistance sensor; the second electronic device is communicatively coupled to the first electronic device by a personal area network protocol; the second electronic device is a smart watch, smart phone, or tablet configured with the physiological sensor; and the behavior-modification system is used to treat an indication selected from depression, anger, angina, anxiety, a stress-related disorder, asthma, emphysema, panic attacks, sleep apnea, and phobias.

In view of the foregoing, a solution for controlling and managing blood pressure and depression to improve health and wellness is provided herein in some embodiments of systems and methods. Systems can include a smart watch or some other smart device with integrated or external physiological sensors like a heart rate monitor worn by an individual configured to receive alerts of various physiological measurements for the individual that indicate periods of stress or relaxation. Methods includes breathing exercises and customizable alerts that allow the user to become aware of situations that cause stress and allow him/her to reduce physiological arousal and lower blood pressure by engaging in breathing and other relaxation exercises. Using a network-connected blood pressure device the user can able to see improvements and have their data remotely monitored by healthcare professionals.

The detailed description is not exclusive of other embodiments and versions and is not limited to any form factor and telecommunication protocol. Its method exclusively restricts its use for biofeedback and auto-regulation for health and wellness improvement and for the management of hypertension and other acute and chronic diseases.

While some particular embodiments have been provided herein, and while the particular embodiments have been provided in some detail, it is not the intention for the particular embodiments to limit the scope of the concepts presented herein. Additional adaptations and/or modifications can appear to those of ordinary skill in the art, and, in broader aspects, these adaptations and/or modifications are encompassed as well. Accordingly, departures may be made from the particular embodiments provided herein without departing from the scope of the concepts provided herein.

What is claimed is:

1. A method, comprising:
setting a physiological threshold value in a user interface for a user behavior-modification system,
wherein at least a portion of the user behavior-modification system is resident in a memory of a first network-connected electronic device configured with a physiological sensor, and
wherein the physiological sensor is configured to physiologically couple to the user for monitoring a physiological parameter of the user;
monitoring the physiological parameter corresponding to the physiological threshold value to alert the user when the user exceeds the physiological threshold value; and
notifying the user with a notification when the user exceeds the physiological threshold value in an event, thereby enabling the user to modify behavior to bring the physiological parameter below the physiological threshold value,
wherein the notification is provided through a vibrator, a speaker, or a display screen of the first electronic device or a second network-connected electronic device.

2. The method of claim 1,
wherein at least a portion of the user behavior-modification system is resident in a memory of a network-connected server computer and a memory of a network-connected client computer, and
wherein the server computer is configured to mediate network communication between a) the client computer and the first electronic device or b) the client computer and the second electronic device such that the user can effect setting the physiological threshold value in the behavior-modification system through an electronic-device user interface or a client-computer user interface.

3. The method of claim 1,
wherein at least a portion of the user behavior-modification system is resident in a memory of a network-connected server computer and a memory of a network-connected client computer, and
wherein the server computer is configured to mediate network communication between a) the client computer and the first electronic device or b) the client computer and the second electronic device such that a healthcare professional can effect setting the physiological threshold value for the user in the behavior-modification system.

4. The method of claim 1, further comprising:
providing instructions to the user on how to bring the physiological parameter below the physiological threshold value,
wherein the instructions are packaged with the notification or provided subsequent to the notification upon a user request for the instructions or a user confirmation from a prompt for the instructions.

5. The method of claim 1, further comprising:
providing a training plan to the user on how to maintain the physiological parameter below the physiological threshold value,
wherein the training plan is packaged with the notification or provided subsequent to the notification upon a user request for the training plan or a user confirmation from a prompt for the training plan.

6. The method of claim 5, further comprising:
providing a training module to the user in accordance with the training plan to aid the user in training to maintain the physiological parameter below the physiological threshold value,
wherein the training modules include at last one chart for the physiological parameter vs. time to aid the user in visualizing training effects on the physiological parameter.

7. The method of claim 1, further comprising:
providing a self-assessment questionnaire to the user; and
providing instructions to the user on how to bring the physiological parameter below the physiological threshold value based upon answers provided by the user on the self-assessment questionnaire.

8. The method of claim 1,
wherein the behavior-modification system includes tagging, thereby enabling the user to tag the event in which the user exceeds the physiological threshold value with contextual information for the event for ongoing monitoring.

9. The method of claim 1,
wherein the first electronic device is a physiological sensor device configured with a sensor selected from the group consisting of an electroencephalogram (EEG) sensor, electromyography (EMG) sensor, an electrocardiography (ECG) sensor, a heart rate variability (HRV) sensor, a respiratory sinus arrhythmia (RSA) sensor, a respiration sensor, a blood pressure sensor, a temperature sensor, a peripheral saturation of oxygen (SpO₂) sensor, weight sensor, and a skin conductance/resistance sensor,
wherein the second electronic device is communicatively coupled to the first electronic device by a personal area network protocol, and
wherein the second electronic device is a smart watch, smart phone, or tablet configured with the physiological sensor.

10. The method of claim 1,
wherein the first electronic device is a smart watch, smart phone, or tablet configured with the physiological sensor.

11. The method of claim 1,
wherein behavior-modification system is used to treat an indication selected from depression, anger, angina, anxiety, a stress-related disorder, asthma, emphysema, hypertension, panic attacks, sleep apnea, and phobias.

12. A method, comprising:
setting a physiological threshold value in a user interface for a user behavior-modification system,
wherein at least a portion of the user behavior-modification system is resident in a memory of a first network-connected electronic device, a memory of a second network-connected electronic device, a memory of a network-connected server computer, and a memory of a network-connected client computer,
wherein the server computer is configured to mediate network communication between a) the client computer and the first electronic device or b) the client computer and the second electronic device such that,
the user can effect setting the physiological threshold value in the behavior-modification system through an electronic-device user interface,
a healthcare professional can effect setting the physiological threshold value in the behavior-modification system through a client-computer user interface, and
wherein the first electronic device is configured with a physiological sensor for physiologically coupling to the user for monitoring a physiological parameter of the user;
monitoring the physiological parameter corresponding to the physiological threshold value to alert the user when the user exceeds the physiological threshold value;
notifying the user with a notification when the user exceeds the physiological threshold value in an event, thereby enabling the user to modify behavior to bring the physiological parameter below the physiological threshold value,
wherein the notification is provided through a vibrator, a speaker, or a display screen of the second electronic device; and
providing instructions to the user on how to bring the physiological parameter below the physiological threshold value,
wherein the instructions are packaged with the notification or provided subsequent to the notification upon a user request for the instructions or a user confirmation from a prompt for the instructions.

13. A non-transient computer-readable medium containing instructions that when executed by a first network-connected electronic device cause the first electronic device to perform a method, comprising:
setting a physiological threshold value in a user interface for a user behavior-modification system,
wherein at least a portion of the user behavior-modification system is resident in a memory of the first electronic device configured with a physiological sensor, and
wherein the physiological sensor is configured to physiologically couple to the user for monitoring a physiological parameter of the user;
monitoring the physiological parameter corresponding to the physiological threshold value to alert the user when the user exceeds the physiological threshold value; and
notifying the user with a notification when the user exceeds the physiological threshold value in an event, thereby enabling the user to modify behavior to bring the physiological parameter below the physiological threshold value,
wherein the notification is provided through a vibrator, a speaker, or a display screen of the first electronic device or a second network-connected electronic device.

14. The non-transient computer-readable medium of claim 13,
wherein at least a portion of the user behavior-modification system is resident in a memory of a network-connected server computer and a memory of a network-connected client computer, and
wherein the server computer is configured to mediate network communication between a) the client computer and the first electronic device or b) the client computer and the second electronic device such that,
the user can effect setting the physiological threshold value in the behavior-modification system through an electronic-device user interface, and
a healthcare professional can effect setting the physiological threshold value in the behavior-modification system through a client-computer user interface.

15. The non-transient computer-readable medium of claim 13, further comprising:
providing instructions to the user on how to bring the physiological parameter below the physiological threshold value,
wherein the instructions are packaged with the notification or provided subsequent to the notification upon a user request for the instructions or a user confirmation from a prompt for the instructions.

16. The non-transient computer-readable medium of claim 13, further comprising:
providing a training plan to the user on how to maintain the physiological parameter below the physiological threshold value,
wherein the training plan is packaged with the notification or provided subsequent to the notification upon a user request for the training plan or a user confirmation from a prompt for the training plan.

17. The non-transient computer-readable medium of claim 16, further comprising:
providing a training module to the user in accordance with the training plan to aid the user in training to maintain the physiological parameter below the physiological threshold value,
wherein the training modules include at last one chart for the physiological parameter vs. time to aid the user in visualizing training effects on the physiological parameter.

18. The non-transient computer-readable medium of claim 13, further comprising:
providing a self-assessment questionnaire to the user; and
providing instructions to the user on how to bring the physiological parameter below the physiological threshold value based upon answers provided by the user on the self-assessment questionnaire.

19. The non-transient computer-readable medium of claim 13,
wherein the behavior-modification system includes tagging, thereby enabling the user to tag the event in which the user exceeds the physiological threshold value with contextual information for the event for ongoing monitoring.

20. The non-transient computer-readable medium of claim 13,
wherein the first electronic device is a physiological sensor device configured with a sensor selected from the group consisting of an electroencephalogram (EEG) sensor, electromyography (EMG) sensor, an electrocardiography (ECG) sensor, a heart rate variability (HRV) sensor, a respiratory sinus arrhythmia (RSA) sensor, a respiration sensor, a blood pressure sensor, a temperature sensor, a peripheral saturation of oxygen (SpO₂) sensor, weight sensor, and a skin conductance/resistance sensor,
wherein the second electronic device is communicatively coupled to the first electronic device by a personal area network protocol,
wherein the second electronic device is a smart watch, smart phone, or tablet configured with the physiological sensor, and
wherein behavior-modification system is used to treat an indication selected from depression, anger, angina, anxiety, a stress-related disorder, asthma, emphysema, panic attacks, sleep apnea, and phobias.
